(19) United States
(12) Patent Application Publication
(10) Pub. No.: US 2026/0346000 A1
(43) Pub. Date: Nov. 5, 2026

(54) BURR HOLE PLUGS WITH LEAD RETENTION ARRANGEMENTS AND METHODS OF MAKING AND USING

(71) Applicant: Boston Scientific Neuromodulation Corporation, Valencia, CA (US)

(72) Inventors: Patrick Michael Marek, Ventura, CA (US); Emma Katherine Koch, Santa Monica, CA (US); Preksha Dheri, Glendale, CA (US)

(21) Appl. No.: 19/662,455

(22) Filed: Apr. 29, 2026

Related U.S. Application Data
(60) Provisional application No. 63/800,309, filed on May 5, 2025.

(51) Int. Cl. A61N 1/05

(52) U.S. Cl. CPC A61N 1/0539

(57) ABSTRACT

A burr hole plug includes a plug base; a retainer for disposition in the plug base, the retainer including a disc defining a lead slot for receiving one or more leads and a clamping surface facing the lead slot, a clamping bar disposed within the lead slot opposite the clamping surface, a threaded rod coupled to the clamping bar, and a first gear including a threaded aperture receiving a portion of the threaded rod; and a cover to be disposed over the retainer and removably coupled to the plug base for lead retention. Rotation of the first gear moves the threaded rod and clamping bar laterally toward or away from the clamping surface of the disc. In some instances, the first gear and threaded rod are replaced by a linear gear or by a screw.

### CROSS-REFERENCE TO RELATED APPLICATIONS

This application claims the benefit under 35 U.S.C. § 119(e) of U.S. Provisional Patent Application Ser. No. 63/800,309, filed May 5, 2025, which is incorporated herein by reference.

### FIELD

The present disclosure is directed to the area of implantable stimulation systems and methods of making and using the systems. The present disclosure is also directed to burr hole plugs and implantable stimulation systems including the burr hole plugs, as well as methods of making and using the burr hole plugs and stimulation systems.

### BACKGROUND

Implantable electrical and optical stimulation systems have proven therapeutic in a variety of diseases and disorders. For example, stimulation of the brain, such as deep brain stimulation, can be used to treat a variety of diseases or disorders, and spinal cord stimulation systems have been used as a therapeutic modality for the treatment of chronic pain syndromes. Peripheral nerve stimulation has been used to treat incontinence, as well as a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the brain, nerves, or other tissue to be stimulated. The pulse generator in the control module generates electrical pulses that are delivered by the electrodes to body tissue.

### BRIEF SUMMARY

One aspect is a burr hole plug that includes a plug base configured for mounting in a burr hole in a skull of a patient; a retainer configured for disposition in the plug base, the retainer including a disc defining a lead slot configured for receiving one or more leads and a clamping surface facing the lead slot, a clamping bar disposed within the lead slot opposite the clamping surface, a threaded rod coupled to the clamping bar, and a first gear including a threaded aperture receiving a portion of the threaded rod, wherein the retainer is configured so that rotation of the first gear moves the threaded rod and clamping bar laterally toward or away from the clamping surface of the disc; and a cover configured to be disposed over the retainer and removably coupled to the plug base for lead retention.

In at least some aspects, the burr hole plug further includes a second gear engaging the first gear and configured for rotating the first gear as the second gear is rotated. In at least some aspects, the second gear is arranged perpendicular to the first gear. In at least some aspects, the second gear is configured for engagement by a tool for rotating the second gear.

Another aspect is a burr hole plug kit that includes the burr hole plug described above and a tool configured for rotating the first gear. In at least some aspects, the tool includes a second gear configured for engaging and rotating the first gear.

Yet another aspect is a method for clamping at least one lead within the burr hole plug described above. The method includes positioning the plug base in the burr hole of the skull of the patient with the at least one lead extending out of the burr hole; disposing the retainer in the plug base with the at least one lead extending through the lead slot defined by the disc of the retainer; and rotating the first gear to move the clamping bar laterally toward the clamping surface of the disc of the retainer to clamp the at least one lead between the clamping bar and the clamping surface.

A further aspect is a burr hole plug that includes a plug base configured for mounting in a burr hole in a skull of a patient; a retainer configured for disposition in the plug base, the retainer including a disc defining a lead slot configured for receiving one or more leads and a clamping surface facing the lead slot, a clamping bar disposed within the lead slot opposite the clamping surface, and a linear gear coupled to the clamping bar, wherein the linear gear is configured for engaging a circular gear, wherein the retainer is configured so that rotation of the circular gear moves the linear gear and clamping bar laterally toward or away from the clamping surface of the disc; and a cover configured to be disposed over the retainer and removably coupled to the plug base for lead retention.

In at least some aspects, the burr hole plug further includes the circular gear engaging the linear gear. In at least some aspects, the circular gear is configured for engagement by a tool for rotating the circular gear.

Another aspect is a burr hole plug kit that includes the burr hole plug described above and a tool configured for rotating the first gear. In at least some aspects, the tool includes the circular gear configured for engaging the linear gear.

Yet another aspect is a method for clamping at least one lead within the burr hole plug described above. The method includes positioning the plug base in the burr hole of the skull of the patient with the at least one lead extending out of the burr hole; disposing the retainer in the plug base with the at least one lead extending through the lead slot defined by the disc of the retainer; engaging the linear gear of the retainer with the circular gear; and rotating the circular gear to move the linear gear and clamping bar laterally toward the clamping surface of the disc of the retainer to clamp the at least one lead between the clamping bar and the clamping surface.

A further aspect is a burr hole plug that includes a plug base configured for mounting in a burr hole in a skull of a patient; a retainer configured for disposition in the plug base, the retainer including a disc defining a lead slot configured for receiving one or more leads and a clamping surface facing the lead slot, a clamping bar disposed within the lead slot opposite the clamping surface, and a threaded screw coupled to the clamping bar, wherein the retainer is configured so that rotation of the screw moves the clamping bar laterally toward or away from the clamping surface of the disc; and a cover configured to be disposed over the retainer and removably coupled to the plug base for lead retention.

In at least some aspects, the retainer further includes a disc tab extending from the disc and a clamp tab extending from the clamping bar, wherein the threaded screw extends from the disc tab to the clamp tab. In at least some aspects, the disc tab defines a non-threaded aperture and the clamp tab defines a threaded aperture, wherein the threaded screw extends through the non-threaded aperture of the disc tab and the threaded aperture of the clamp tab.

In at least some aspects, the clamping bar includes a threaded aperture and the threaded screw extends into the threaded aperture of the clamping bar. In at least some aspects, the retainer further includes a screw tab extending from the disc, wherein the screw tab includes a non-threaded aperture and the screw extends through the non-threaded aperture of the screw tab.

Another aspect is a burr hole plug kit that includes the burr hole plug described above and a tool configured for rotating the screw.

Yet another aspect is a method for clamping at least one lead within the burr hole plug described above. The method includes positioning the plug base in the burr hole of the skull of the patient with the at least one lead extending out of the burr hole; disposing the retainer in the plug base with the at least one lead extending through the lead slot defined by the disc of the retainer; and rotating the screw to move the clamping bar laterally toward the clamping surface of the disc of the retainer to clamp the at least one lead between the clamping bar and the clamping surface.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system;

FIG. 2 is a schematic side view of one embodiment of an electrical stimulation lead;

FIG. 3 is an exploded perspective view of components of one embodiment of a known burr hole plug;

FIG. 4A is a top perspective view of a retainer of the burr hole plug of FIG. 3;

FIG. 4B is a bottom perspective view of the retainer of the burr hole plug of FIG. 3;

FIG. 5A is a top view of a first embodiment of a retainer of a burr hole plug with a two-gear mechanism for moving a clamping bar;

FIG. 5B is a side perspective view of the retainer of FIG. 5A;

FIG. 5C is another side perspective view of the retainer of FIG. 5A with the clamping bar partially extended into a lead slot;

FIG. 5D is side view of one embodiment of a tool for use with the retainers disclosed herein;

FIG. 6A is a side perspective view of a second embodiment of a retainer of a burr hole plug with a geared mechanism for moving a clamping bar using a tool with a gear;

FIG. 6B is a side perspective view of the retainer of FIG. 6A with the tool no longer in contact with the retainer;

FIG. 7A is a top view of a third embodiment of a retainer of a burr hole plug with a rack-and-pinion mechanism (e.g., a mechanism with linear and rotational gears) for moving a clamping bar;

FIG. 7B is a side perspective view of the retainer of FIG. 7A;

FIG. 7C is another side perspective view of the retainer of FIG. 7A with the clamping bar partially extended into a lead slot;

FIG. 8A is a top view of a fourth embodiment of a retainer of a burr hole plug with linear gear mechanism for moving a clamping bar using a tool with a rotational gear;

FIG. 8B is a side perspective view of the retainer of FIG. 8A with the tool engaged with the retainer;

FIG. 8C is a side perspective view of the retainer of FIG. 8A with the tool no longer in contact with the retainer;

FIG. 9A is a top view of a fifth embodiment of a retainer of a burr hole plug with a screw mechanism for moving a clamping bar;

FIG. 9B is a side perspective view of the retainer of FIG. 9A;

FIG. 9C is another side perspective view of the retainer of FIG. 9A with the clamping bar partially extended into a lead slot;

FIG. 10A is a top view of a sixth embodiment of a retainer of a burr hole plug with a screw mechanism for moving a clamping bar;

FIG. 10B is a side perspective view of the retainer of FIG. 10A;

FIG. 10C is another side perspective view of the retainer of FIG. 10A with the clamping bar partially advanced into a lead slot;

FIG. 10D is side perspective view from a different angle of the retainer of FIG. 10A with the clamping bar partially advanced into the lead slot; and

FIG. 11 is a side view of one embodiment of a ratchet mechanism for use with any of the retainer disclosed herein.

### DETAILED DESCRIPTION

The present disclosure is directed to the area of implantable stimulation systems and methods of making and using the systems. The present disclosure is also directed to burr hole plugs and implantable electrical stimulation systems including the burr hole plugs, as well as methods of making and using the burr hole plugs and stimulation systems.

Suitable implantable electrical stimulation systems include, but are not limited to, at least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; 8,473,061; 8,571,665; 8,688,235; and 8,792,993; and U.S. Patent Applications Publication Nos. 2007/0150036; 2009/0187222; 2009/0276021; 2010/0076535; 2010/0268298; 2011/0005069; 2011/0004267; 2011/0078900; 2011/0130803; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2013/0197602; 2013/0197424; 2013/0197602; 2014/0039587; 2014/0353001; 2014/0358208; 2014/0358209; 2014/0358210; 2015/0045864; 2015/0066120; 2015/0018915; and 2015/0051681, all of which are incorporated by reference.

In the discussion below, electrical stimulation leads and electrical stimulation systems are exemplified, but it will be understood that optical stimulation leads, optical/electrical stimulation leads, and other leads can be used, as well as optical stimulation systems and optical/electrical stimulation systems. Examples of optical stimulation or modulation systems or electrical/optical stimulation systems, which include one or more optical emitters in addition to, or as an alternative to, electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties.

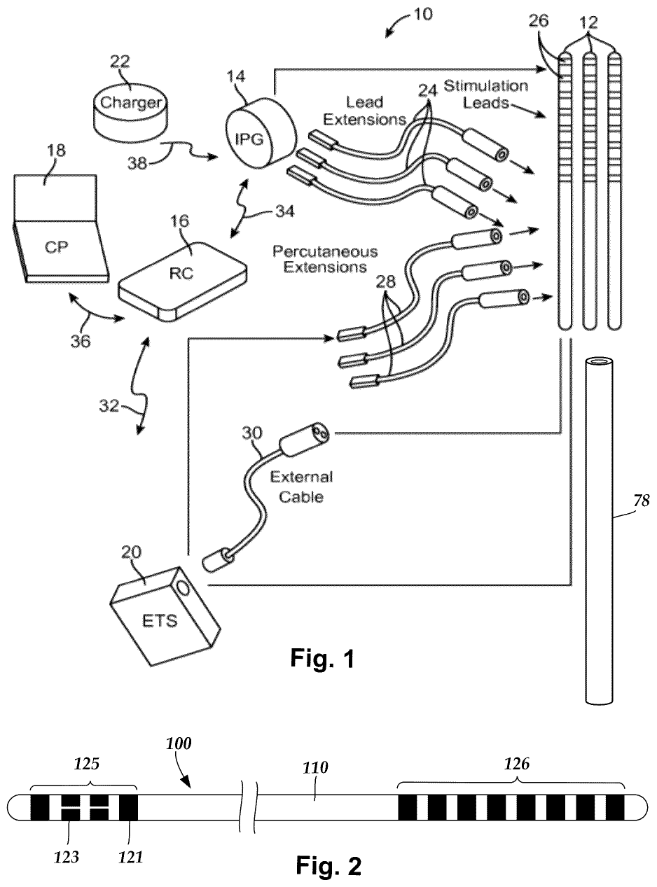

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The stimulation lead(s) 12 can be implanted into the patient using an insertion tool 78, such as a cannula, needle, or the like.

The IPG 14 is physically connected, optionally, via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's abdominal cavity. The implantable pulse generator can have eight stimulation channels which may be independently programmable to control the magnitude of the current stimulus from each channel. In some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated by reference.

Turning to FIG. 2, one or more leads are configured for coupling with a control module. The term “control module” is used herein to describe a pulse generator (e.g., the IPG 14 or the ETS 20 of FIG. 1). Stimulation signals generated by the control module are emitted by electrodes of the lead(s) to stimulate patient tissue. The electrodes of the lead(s) are electrically coupled to terminals of the lead(s) that, in turn, are electrically coupleable with the control module. In some embodiments, the lead(s) couple(s) directly with the control module. In other embodiments, one or more intermediary devices (e.g., a lead extension, an adaptor, a splitter, or the like) are disposed between the lead(s) and the control module.

FIG. 2 illustrates one embodiment of a lead 100 (corresponding to lead 12 of FIG. 1) with a lead body 110, electrodes 125 disposed at least partially about a circumference of the lead 100 along a distal end portion of the lead, and terminals 126 disposed along a proximal end portion of the lead. The lead 100 can be implanted near or within the desired portion of the body to be stimulated such as, for example, the brain. In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 100 can be inserted into the cranium and brain tissue with the assistance of a stylet (not shown). The lead 100 can be guided to the target location within the brain using, for example, a stereotactic frame and a microdrive motor system. In some embodiments, the microdrive motor system can be fully or partially automatic. The microdrive motor system may be configured to perform one or more of the following actions (alone or in combination): insert the lead 100, advance the lead 100, retract the lead 100, or rotate the lead 100.

The lead 100 for deep brain stimulation can include stimulation electrodes, recording electrodes, or both. Stimulation electrodes may be disposed on the circumference of the lead 100 to stimulate the target neurons. Stimulation electrodes may be ring-shaped so that current projects from each electrode equally in every direction from the position of the electrode along a length of the lead 100. In the embodiment of FIG. 2, two of the electrodes 125 are ring electrodes 121. Segmented electrodes 123 can be used to direct stimulus current to a selected angular range around the lead 100. When segmented electrodes 123 are used in conjunction with an implantable pulse generator that delivers constant current stimulus, current steering can be achieved to deliver the stimulus more precisely to a position around an axis of the lead (i.e., radial positioning around the axis of the lead 100). To achieve current steering, segmented electrodes 123 can be utilized in addition to, or as an alternative to, ring electrodes 121.

The lead body 100 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 100 may be in contact with body tissue for extended periods of time. In at least some embodiments, the lead 100 has a cross-sectional diameter of no more than 1.5 mm and may be in the range of 0.5 to 1.5 mm. In at least some embodiments, the lead 100 has a length of at least 10 cm and the length of the lead 100 may be in the range of 10 to 70 cm.

The electrodes 125 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use. Each of the electrodes can either be used (ON) or unused (OFF). When the electrode is used, the electrode can be used as an anode or cathode and carry anodic or cathodic current. In some instances, an electrode might be an anode for a period of time and a cathode for a period of time.

In at least some instances of stimulation of the brain, when one or more leads are implanted into the brain of a patient, each lead is inserted through a burr hole in the skull of the patient. Each lead extends out of the burr hole and is coupled to a control module implanted elsewhere, for example, in the torso of the patient. A burr hole plug is provided in the burr hole to cover the opening through the skull, to protect the lead exiting the skull, and to firmly hold the lead(s) in place to prevent or reduce lead migration within the brain.

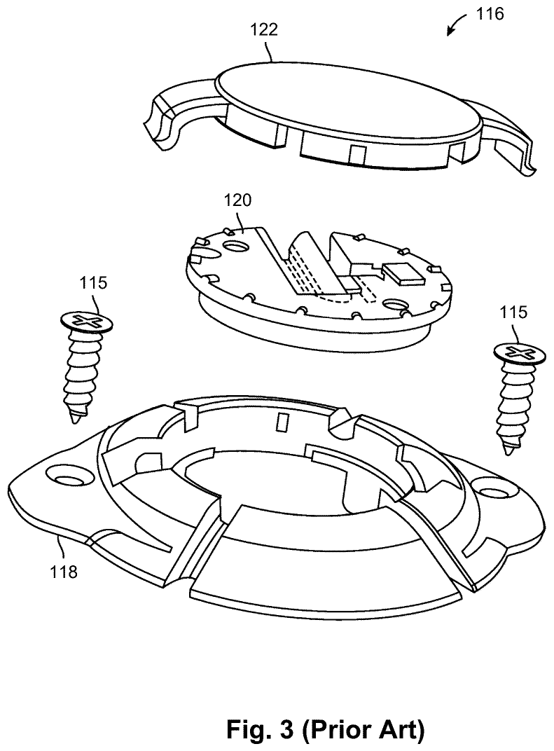

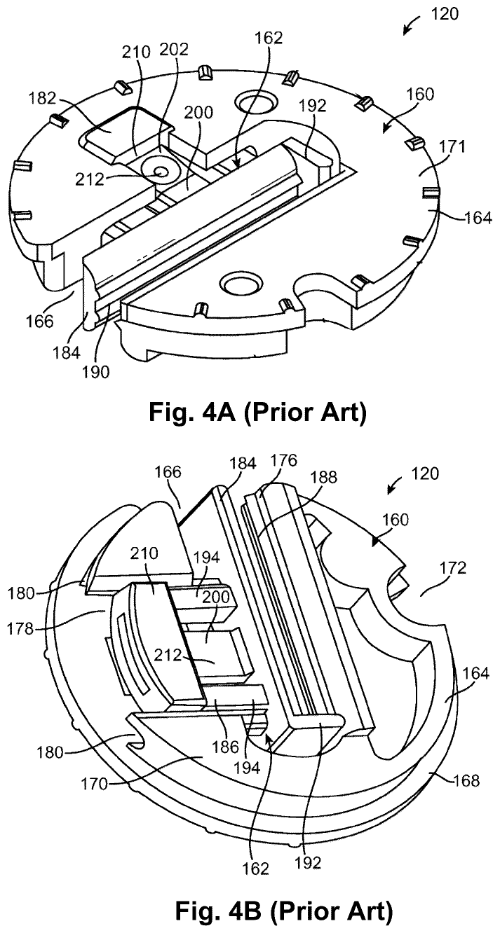

FIGS. 3, 4A, and 4B illustrate one embodiment of a burr hole plug described in U.S. Pat. No. 9,043,000, incorporated herein by reference in its entirety. As described below, this burr hole plug can be modified to provide alternative methods and mechanisms for operation of a clamping bar. Other examples of burr hole plugs that can be modified with the alternative methods and mechanism presented below include the burr hole plugs described in U.S. Pat. Nos. 8,024,045; 8,043,304; 8,425,534; 8,670,839; 8,731,686; 8,812,133; 9.474,896; 10,232,169; 11,013,913; and 11,058,870 and U.S. patent application Ser. No. 18/935,291, all of which are incorporated herein by reference in their entireties. It will be understood that the methods and mechanisms described herein can be used in other burr hole plug designs.

FIG. 3 illustrates a burr hole plug 116 that includes a plug base 118 configured for mounting at a burr hole in a skull of the patient, a retainer 120 for securing at least one stimulation lead extending through the burr hole, a cap 122 for mounting on the plug base over the retainer and closing the burr hole, and fasteners, such as screws 115, for attaching the plug base to the cranium of the patient.

FIGS. 4A and 4B illustrate one embodiment of the retainer 120 of the burr hole plug 116. The retainer 120 includes a retainer support 160 that has a disc 164 and a clamping mechanism 162. The disc 164 has a top surface 171 and a bottom surface 170

The clamping mechanism 162 defines a lead slot 166 and has a movable clamping bar 184 and a flange 186 slidably engaged with the disc 164 to laterally slide the movable clamping bar 184 relative to the disc 164 and secure the stimulation lead received within the lead slot or release the stimulation lead received within the lead slot. The movable clamping bar 184 extends parallel to the lead slot 166 and opposite to a fixed clamping bar 176 on the disc 164, such that a clamping surface 190 of the movable clamping bar 184 is configured for clamping the stimulation lead against a clamping surface 188 of the fixed clamping bar 176. The clamping mechanism 162 also includes an angled flange 192 located at the end of the movable clamping bar 184 to prevent the stimulation lead from being located past the movable clamping bar 184 at the end of the lead slot 166 where it could potentially be wedged between the clamping mechanism 162 and the end of the lead slot.

In the illustrated embodiment, the flange 186 is U-shaped and includes a pair of legs 194 extending perpendicularly from the movable clamping bar 184 away from the lead slot 166. The sliding arrangement between the flange 186 of the clamping mechanism 162 and the disc 164 of the retainer support 160 is provided between the legs 194 of the U-shaped flange 186 and C-channels 180 of the retainer support 160. In particular, the U-shaped flange 186 is received within a recess 178 and within the C-channels 180 so that the flange 186 and the movable clamping bar 184 can be smoothly moved back and forth in a lateral direction (i.e., perpendicular to the lead slot 166).

In the illustrated embodiment, the clamping mechanism 162 includes a resilient arm 200 extending perpendicularly from the movable clamping bar 184 between the legs 194 of the U-shaped flange 186. The clamping mechanism 162 also includes a locking tab 202, with a recess 212, located at the end of the resilient arm 200. The locking tab 202 is configured for engaging a complementary locking element (not shown) on the disc 164 to lock the movable clamping bar 184 relative to the disc 164 when the stimulation lead is secured.

A tool is used to slide the movable clamping bar 184 towards or away from the fixed clamping bar 176 to secure or release the stimulation lead. The clamping mechanism 162 can be moved from an unlocked position (or open position) to a locked position by sliding the U-shaped flange 186 and the movable clamping bar 184 relative to the disc 164 towards the fixed clamping bar 176 as the locking tab 202 slides along a recessed stop 182 until the locking tab 202 reaches an abutment surface 210 of the recessed stop 182.

Described herein are alternative mechanisms and methods for moving the clamping bar 184 relative to the disc 164. Any of the burr hole plugs described in the references cited herein or otherwise known can be modified to include one or more of these mechanisms or methods.

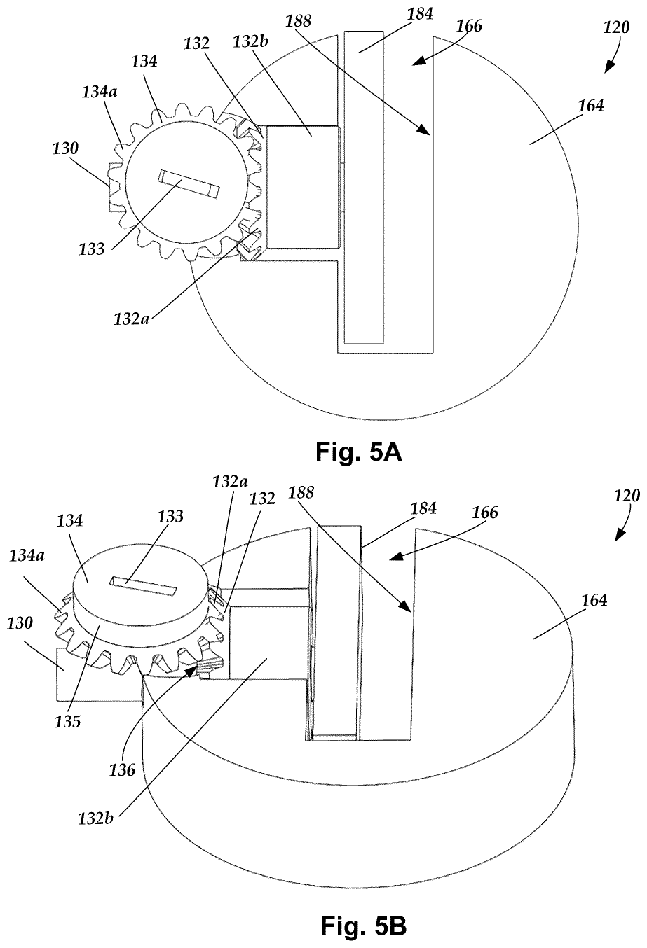

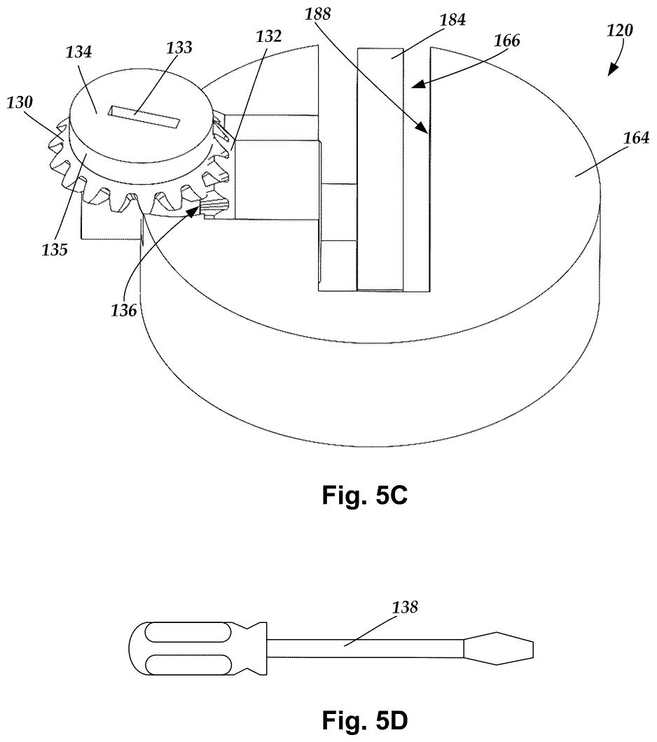

FIGS. 5A to 5C illustrate one embodiment of a retainer 120 that includes a disc 164, a clamping bar 184, a threaded rod 130, a first gear 132, and a second gear 134. In at least some embodiments, the first gear includes teeth 132a and a base 132b and the second gear 134 includes teeth 134a. The threaded rod 130 can be threaded along an entire length of the threaded rod or only along a portion of the length of the threaded rod. In at least some embodiments, the disc 164 includes a structure (for example, a cover over the base 132b of the first gear 132) for retention of the first gear 132 or second gear 134 on the disc 164.

The threaded rod 130 is attached or otherwise coupled to the clamping bar 184 and passes through a threaded aperture 136 (FIG. 5B) in the first gear 132. Rotation of the first gear 132 moves the threaded rod 130 laterally (due to the threading on the threaded rod and within the threaded aperture 136) resulting in corresponding lateral movement of the clamping bar 184, as shown in FIGS. 5B and 5C.

The second gear 134 engages the first gear 132 so that rotation of the second gear 134 results in rotation of the first gear. In at least some embodiments, the first and second gears 132, 134 are beveled gears (e.g., have beveled teeth 132a, 134a, as illustrated in FIG. 5B). In at least some embodiments, the second gear 134 is oriented perpendicularly (i.e., at 90 degrees relative) to the first gear 132. It will be understood that, in other embodiments, the second gear 134 is oriented at an angle in a range of 30 to 90 degrees relative to the first gear 132.

The second gear 134 is configured for rotation using a tool 138 (FIG. 5D), such as a screwdriver, wrench, hex tool, or the like. In at least some embodiments, the second gear 134 includes at least one indentation 133, such as one or more slots or an indentation have a cross-sectional shape of a cross, star, triangle, square, rectangle, pentagon, hexagon, octagon, or any other suitable non-circular shape, for receiving a distal end of the tool 138. In other embodiments, an engagement portion 135 (FIG. 5D) of the second gear 134 has a non-circular shape (e.g., a cross-sectional shape of a triangle, square, rectangle, pentagon, hexagon, octagon, or any other suitable non-circular shape) for engagement by a tool 138, such as a wrench.

A user engages the second gear 134 with the tool 138 and rotates the second gear 134 with the tool 138. Rotation of the second gear 134 rotates the first gear 132 resulting in lateral movement of the threaded rod 130 and the clamping bar 184. Rotation of the second gear 134 in one direction moves the clamping bar 184 towards the clamping surface 188 of the disc 164 for securement of one or more leads 100 extending through the lead slot 166. Rotation of the second gear 134 in the opposite direction moves the clamping bar 184 away from the clamping surface 188 of the disc 164 to release one or more leads 100 extending through the lead slot 166.

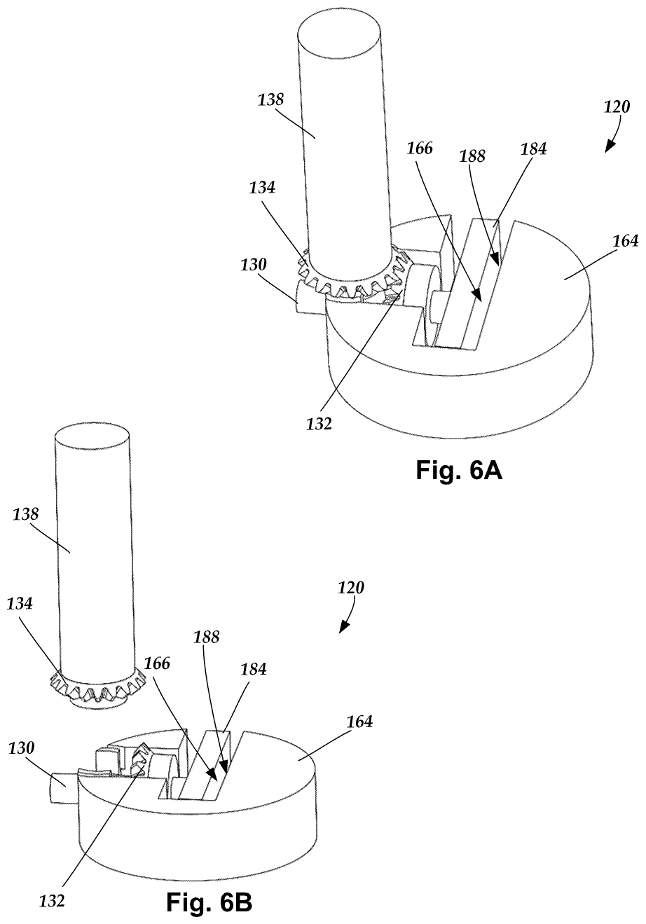

FIGS. 6A and 6B illustrate another embodiment of a retainer 120 that is the same as the embodiment of the retainer 120 presented in FIGS. 5A to 5C except that the second gear 134 is part of the tool 138 instead of part of the retainer 120. The second gear 134 on the tool 138 engages the first gear 132 of the retainer 120 and then the user rotates the tool 138 to rotate the first gear 132 resulting in lateral movement of the threaded rod 130 and the clamping bar 184.

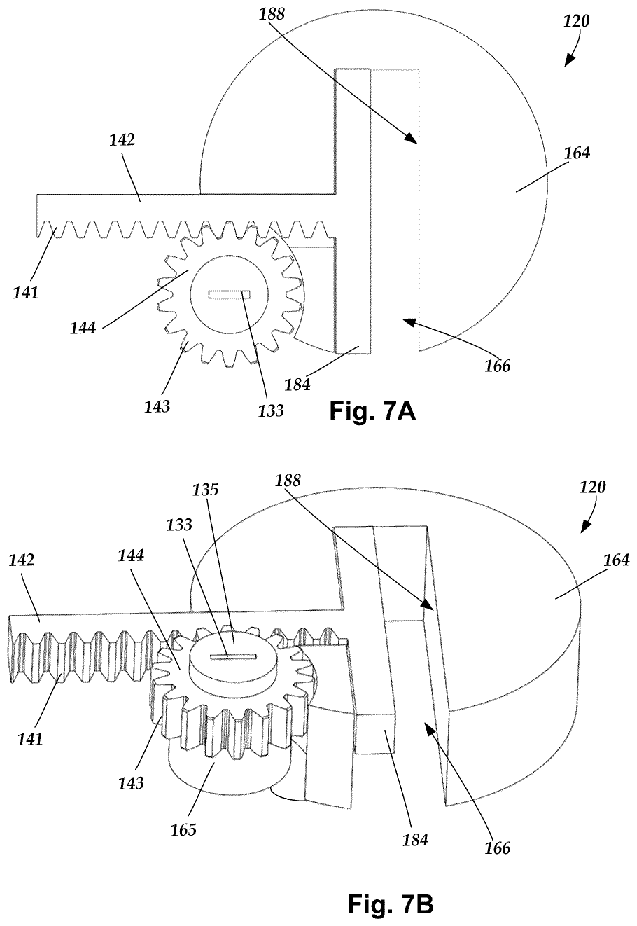

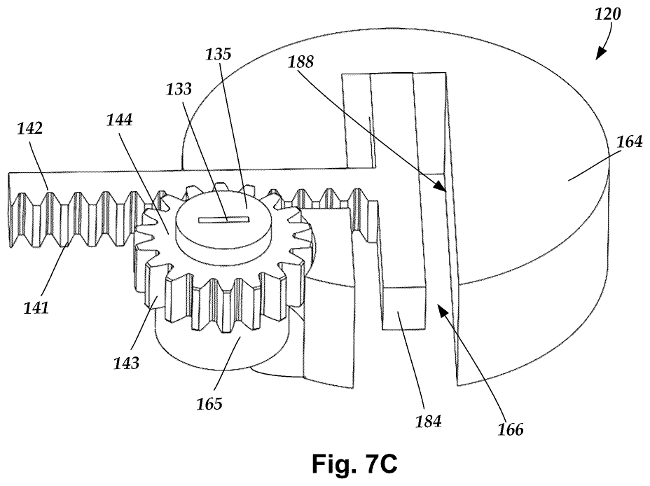

FIGS. 7A to 7C illustrate yet another embodiment of a retainer 120 that includes a disc 164, a clamping bar 184, a linear gear 142 (e.g., a rack), and a circular gear 144 (e.g., a pinion). The linear gear 142 includes gear teeth 141, which may extend along the entire linear gear or only a portion of the linear gear, attached or otherwise coupled to the clamping bar 184. The linear gear 142 and clamping bar 184 can be separate components or can be permanently attached or manufactured as a single unit.

The circular gear 144 includes gear teeth 143 that engage the gear teeth 141 of the linear gear 142 so that rotation of the circular gear 144 results in lateral movement of the linear gear. In at least some embodiments, the circular gear 144 is rotatably attached to the disc 164 or other portion of the retainer 120. In at least some embodiments, the circular gear 144 is rotatably mounted on a post 165 attached to, or otherwise part of, the disc 164 or to another portion of the burr hole plug.

The circular gear 144 is configured for rotation using a tool 138 (FIG. 5D), such as a screwdriver, wrench, hex tool, or the like. In at least some embodiments, the circular gear 144 includes at least one indentation 133, such as one or more slots or an indentation have a cross-sectional shape of a cross, star, triangle, square, rectangle, pentagon, hexagon, octagon, or any other suitable non-circular shape, for receiving a distal end of the tool 138. In other embodiments, an engagement portion 135 (FIG. 7B) has a non-circular shape (e.g., a cross-sectional shape of a triangle, square, rectangle, pentagon, hexagon, octagon, or any other suitable non-circular shape) for engagement by a tool 138, such as a wrench.

A user engages the circular gear 144 with the tool 138 and rotates the circular gear 144 with the tool 138. Rotation of the circular gear 144 results in lateral movement of the linear gear 142 and the clamping bar 184. Rotation of the circular gear 144 in one direction moves the clamping bar 184 towards the clamping surface 188 of the disc 164 for securement of one or more leads 100 extending through the lead slot 166. Rotation of the circular gear 144 in the opposite direction moves the clamping bar 184 away from the clamping surface 188 of the disc 164 to release any lead 100 extending through the lead slot 166.

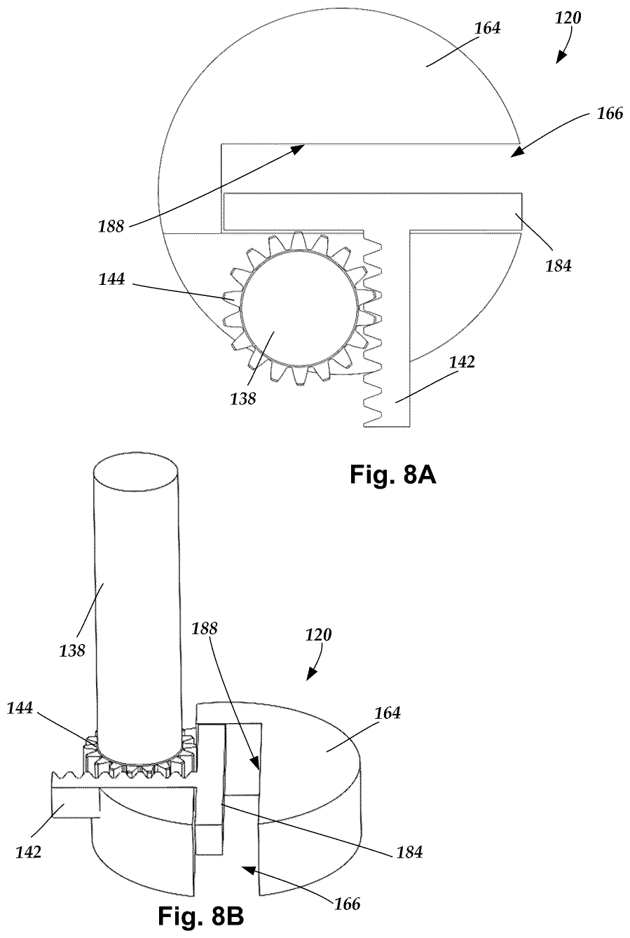

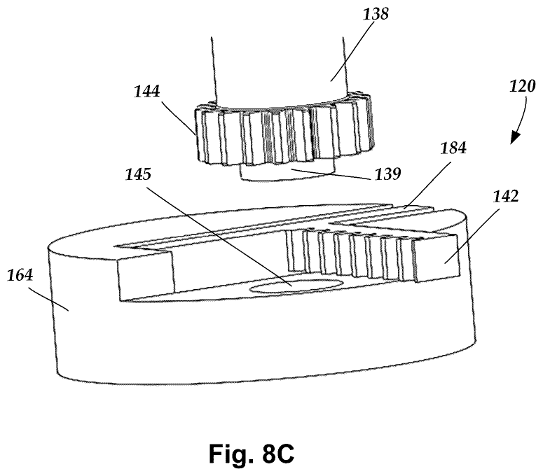

FIGS. 8A to 8C illustrate another embodiment of a retainer 120 that is the same as the embodiment of the retainer 120 presented in FIGS. 7A to 7C except that the circular gear 144 is part of the tool 138 instead of part of the retainer 120. The circular gear 144 on the tool 138 engages the linear gear 142 of the retainer 120 and then the user rotates the tool 138 resulting in lateral movement of the linear gear 142 and the clamping bar 184. In at least some embodiments, as illustrated in FIG. 8C, the disc 164 includes an indentation or opening 145 into which a distal tip 139 of the tool 138 is inserted to align the circular gear 144 with the linear gear 142.

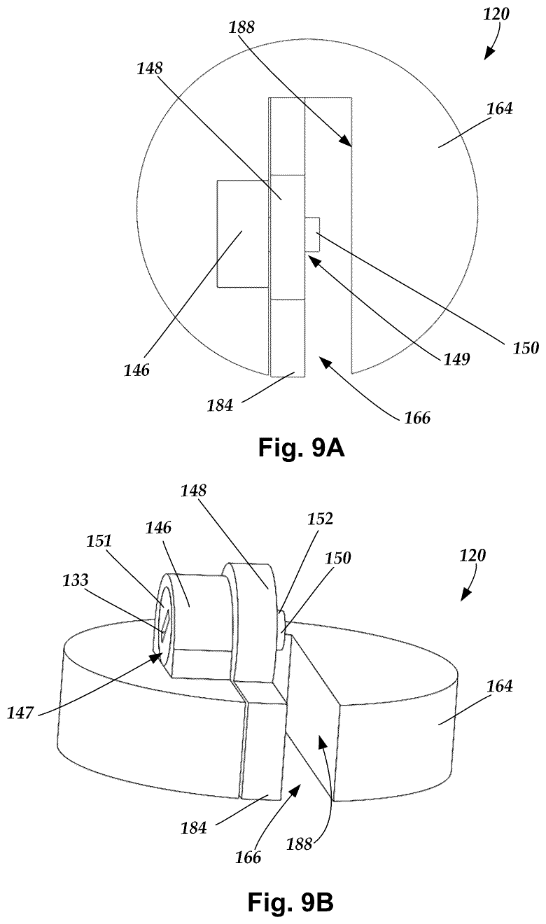

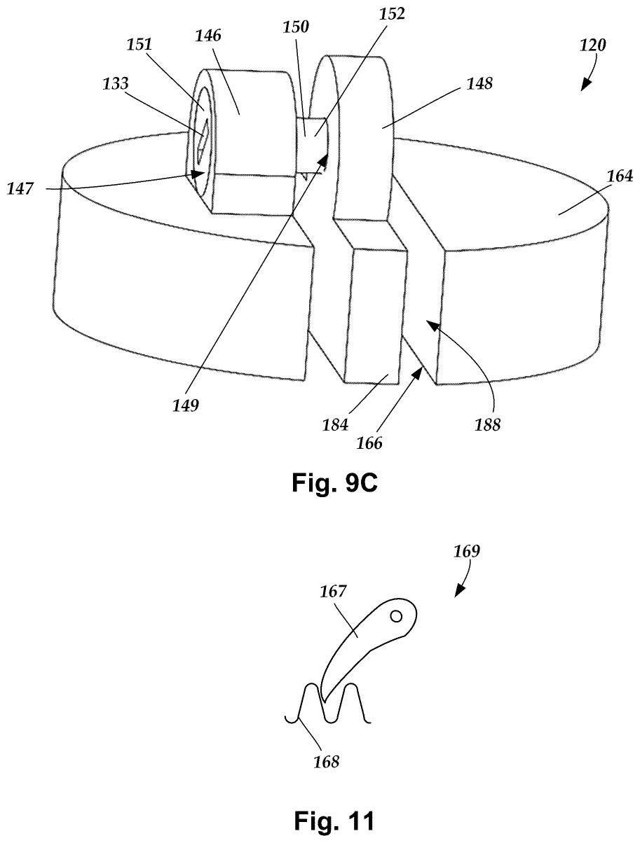

FIGS. 9A to 9C illustrate yet another embodiment of a retainer 120 that includes a disc 164, a clamping bar 184, a disc tab 146, a clamp tab 148, and a screw 150. The screw 150 includes a threaded rod 152 and a head 151 attached to the threaded rod and having a larger diameter than the threaded rod. The threaded rod 152 can be threaded along an entire length of the threaded rod or only along a portion of the length of the threaded rod.

The disc tab 146 extends from the disc 164 and may be a permanent part of the disc (e.g., the disc tab and disc are a single, unitary piece) or attached during or after manufacture of the disc. The clamp tab 148 extends from the clamping bar 184 and may be a permanent part of the clamping bar (e.g., the clamp tab and clamping bar are a single, unitary piece) or attached during or after manufacture of the clamping bar. The disc tab 146 defines a non-threaded aperture 147 and the clamp tab 148 defines a threaded aperture 149. The threaded rod 152 of the screw 150 passes through both the non-threaded aperture 147 of the disc tab 146 and the threaded aperture 149 of the clamp tab 148. In at least some embodiments, the non-threaded aperture 147 of the disc tab 146 is countersunk to receive the head 151 of the screw 150.

The screw 150 is configured for rotation using a tool 138 (FIG. 5D), such as a screwdriver, wrench, hex tool, or the like. In at least some embodiments, the screw 150 includes at least one indentation 133, such as one or more slots or an indentation have a cross-sectional shape of a cross, star, triangle, square, rectangle, pentagon, hexagon, octagon, or any other suitable non-circular shape, for receiving a distal end of the tool 138. In other embodiments, the screw includes an engagement portion that extends beyond the disc tab 146 and has a non-circular shape (e.g., a cross-sectional shape of a triangle, square, rectangle, pentagon, hexagon, octagon, or any other suitable non-circular shape) for engagement by a tool 138, such as a wrench. In at least some embodiments, the tool 138 can be “L-shaped” or have a bend, which may facilitate access to the screw 150.

A user engages the screw 150 with the tool 138 and rotates the screw 150 with the tool 138. Rotation of the screw 150 results in lateral movement of the clamping bar 184 along the threaded rod 152 of the screw due to the threading on the threaded rod 152 and in the threaded aperture 149 of the clamp tab 148. Rotation of the screw 150 in one direction moves the clamping bar 184 towards the clamping surface 188 of the disc 164 for securement of one or more leads 100 extending through the lead slot 166. Rotation of the screw 150 in the opposite direction moves the clamping bar 184 away from the clamping surface 188 of the disc 164 to release any lead 100 extending through the lead slot 166.

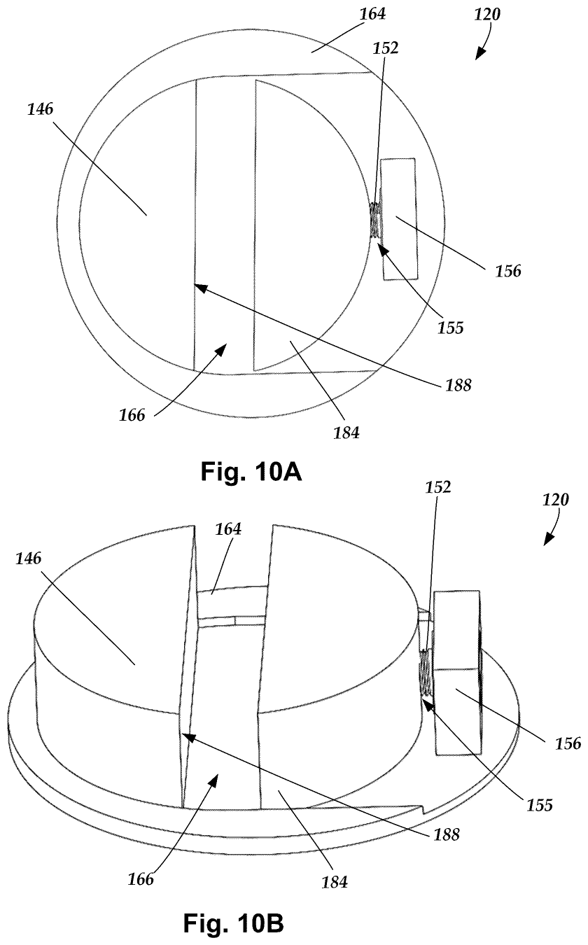

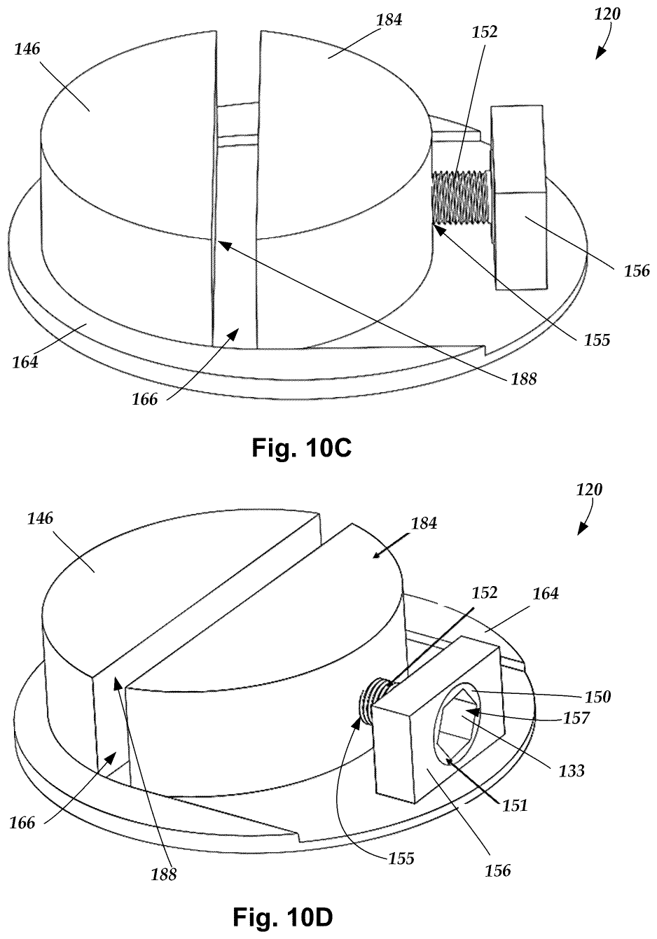

FIGS. 10A to 10C illustrate a further embodiment of a retainer 120 that includes a disc 164, a clamping bar 184, a disc tab 146, a screw 150, and a screw tab 156. The screw tab 156 extends from the disc 164 and may be a permanent part of the disc (e.g., the screw tab and disc are a single, unitary piece) or attached during or after manufacture of the disc. The screw tab 156 includes a non-threaded aperture 157.

The screw 150 includes a threaded rod 152 and a head 151 (FIG. 10D) attached to the threaded rod and having a larger diameter than the threaded rod. The threaded rod 152 can be threaded along an entire length of the threaded rod or only along a portion of the length of the threaded rod. In at least some embodiments, the threaded rod 152 of the screw 150 extends into the clamping bar 184 but no further. In at least some other embodiments, the threaded rod of the screw 150 can extend into the lead slot 166 or into the disc tab 146.

The disc tab 146 extends from the disc 164 and may be a permanent part of the disc (e.g., the disc tab and disc are a single, unitary piece) or attached during or after manufacture of the disc. The disc tab 146 defines a clamping surface 188. The clamping bar 184 defines a threaded aperture 155. The threaded rod 152 of the screw 150 passes through the non-threaded aperture 157 of the screw tab and the threaded aperture 155 of the clamping bar 184.

The screw 150 is configured for rotation using a tool 138 (FIG. 5D), such as a screwdriver, wrench, hex tool, or the like. In at least some embodiments, the screw 150 includes at least one indentation 133, such as one or more slots or an indentation have a cross-sectional shape of a cross, star, triangle, square, rectangle, pentagon, hexagon, octagon, or any other suitable non-circular shape, for receiving a distal end of the tool 138. In other embodiments, the screw includes an engagement portion that extends beyond the disc tab 146 and has a non-circular shape (e.g., a cross-sectional shape of a triangle, square, rectangle, pentagon, hexagon, octagon, or any other suitable non-circular shape) for engagement by a tool 138, such as a wrench. In at least some embodiments, the tool 138 can be “L-shaped” or have a bend, which may facilitate access to the screw 150.

A user engages the screw 150 with the tool 138 and rotates the screw 150 with the tool 138. Rotation of the screw 150 results in lateral movement of the clamping bar 184 along the threaded rod 152 of the screw due to the threading on the threaded rod 152 and in the threaded aperture 155 of the clamping bar 184. Rotation of the screw 150 in one direction moves the clamping bar 184 towards the clamping surface 188 of the disc tab 146 and securement of one or more leads 100 extending through the lead slot 166. Rotation of the screw 150 in the opposite direction moves the clamping bar 184 away from the clamping surface 188 of the disc tab 146 to release any lead 100 extending through the lead slot 166.

For any of the embodiments described herein, one or both of the clamping bar 184 and clamping surface 188 can include surface features such as, for example, a roughened surface, surface texture, or one or more ribs, teeth, dimples, bumps, serrations, other protrusions, or the like for gripping the lead(s) when the clamping bar is closed toward the clamping surface. In at least some embodiments, the surface features are arranged to prevent or reduce lateral movement or vertical movement (or both) of the lead(s) within the lead slot 166 when clamped (e.g., engaged by both the clamping bar 184 and clamping surface 188).

For any of the embodiments described above, the tool 138 is preferably not implanted with the retainer 120.

Any of the embodiments described herein can include a mechanism 169 such as a locking mechanism or a mechanism to prevent or hinder movement of the clamping bar 184 away from the clamping surface 188 after clamping the lead(s). Examples of such mechanisms include, but are not limited to, ratcheting mechanisms, annular joints, snap fit joints, cantilevered tabs, or the like or any combination thereof. For example, in at least some embodiments, such a mechanism 169 can include, for example, a ratchet pawl 167 can engage teeth/threading 168 of any one of the first gear 132, second gear 134, threaded rod 130, linear gear 142, circular gear 144, or threaded rod 152 of the retainer embodiments as illustrated in FIG. 11. In other embodiments, a threaded rod 130, threaded rod 152, screw 150, or one or more surface features of the disc 164 or clamping bar 184 can form an annular joint or snap fit joint with the first gear 132, clamp tab 148, clamping bar 184, disc tab 146, or one or more surface features on an opposing surface of the disc 164 or clamp tab 148. In yet other embodiments, the threaded rod 130, threaded rod 152 or screw 150 can include a cantilever tab that, when released from disposition within the clamping bar as the clamping bar 184 or clamp tab 148 moves along the threaded rod or screw, will extend away from the threaded rod or screw to resist or hinder movement of the clamping bar 184 away from the clamping surface 188. Any other suitable mechanism for preventing or hindering movement of the clamping bar 184 away from the clamping surface 188 after clamping the lead(s) can be used.

In at least some embodiments, any of the burr hole plugs 116 extend no more than 4, 4.5, 5, 6, 8, or 10 mm above the skull of the patient. In at least some embodiments, any of the burr hole plugs 116 has a maximum width or diameter that is no more than 20, 25, 30, 35, 40, or 50 mm. In at least some embodiments, the burr hole aperture formed in the skull of the patient has a maximum width or diameter of no more than 10, 12, 14, 15, 18, 20, 25, or 30 mm. The components of any of the burr hole plugs 116 or retainers 120 can be made of any suitable material including, but not limited to, rigid plastics, metals, alloys, or any other suitable biocompatible material.

The above specification, examples and data provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A burr hole plug, comprising:
a plug base configured for mounting in a burr hole in a skull of a patient;
a retainer configured for disposition in the plug base, the retainer comprising a disc defining a lead slot configured for receiving one or more leads and a clamping surface facing the lead slot, a clamping bar disposed within the lead slot opposite the clamping surface, a threaded rod coupled to the clamping bar, and a first gear comprising a threaded aperture receiving a portion of the threaded rod, wherein the retainer is configured so that rotation of the first gear moves the threaded rod and clamping bar laterally toward or away from the clamping surface of the disc; and
a cover configured to be disposed over the retainer and removably coupled to the plug base for lead retention.

2. The burr hole plug of claim 1, further comprising a second gear engaging the first gear and configured for rotating the first gear as the second gear is rotated.

3. The burr hole plug of claim 2, wherein the second gear is arranged perpendicular to the first gear.

4. The burr hole plug of claim 3, wherein the second gear is configured for engagement by a tool for rotating the second gear.

5. A burr hole plug kit, comprising:
the burr hole plug of claim 1; and
a tool configured for rotating the first gear.

6. The burr hole plug kit of claim 5, wherein the tool comprising a second gear configured for engaging and rotating the first gear.

7. A method for clamping at least one lead within the burr hole plug of claim 1, the method comprising:
positioning the plug base in the burr hole of the skull of the patient with the at least one lead extending out of the burr hole;
disposing the retainer in the plug base with the at least one lead extending through the lead slot defined by the disc of the retainer; and
rotating the first gear to move the clamping bar laterally toward the clamping surface of the disc of the retainer to clamp the at least one lead between the clamping bar and the clamping surface.

8. A burr hole plug, comprising:
a plug base configured for mounting in a burr hole in a skull of a patient;
a retainer configured for disposition in the plug base, the retainer comprising a disc defining a lead slot configured for receiving one or more leads and a clamping surface facing the lead slot, a clamping bar disposed within the lead slot opposite the clamping surface, and a linear gear coupled to the clamping bar, wherein the linear gear is configured for engaging a circular gear, wherein the retainer is configured so that rotation of the circular gear moves the linear gear and clamping bar laterally toward or away from the clamping surface of the disc; and
a cover configured to be disposed over the retainer and removably coupled to the plug base for lead retention.

9. The burr hole plug of claim 8, further comprising the circular gear engaging the linear gear.

10. The burr hole plug of claim 9, wherein the circular gear is configured for engagement by a tool for rotating the circular gear.

11. A burr hole plug kit, comprising:
the burr hole plug of claim 8; and
a tool configured for rotating the first gear.

12. The burr hole plug kit of claim 11, wherein the tool comprising the circular gear configured for engaging the linear gear.

13. A method for clamping at least one lead within the burr hole plug of claim 8, the method comprising:
positioning the plug base in the burr hole of the skull of the patient with the at least one lead extending out of the burr hole;
disposing the retainer in the plug base with the at least one lead extending through the lead slot defined by the disc of the retainer;
engaging the linear gear of the retainer with the circular gear; and
rotating the circular gear to move the linear gear and clamping bar laterally toward the clamping surface of the disc of the retainer to clamp the at least one lead between the clamping bar and the clamping surface.

14. A burr hole plug, comprising:
a plug base configured for mounting in a burr hole in a skull of a patient;
a retainer configured for disposition in the plug base, the retainer comprising a disc defining a lead slot configured for receiving one or more leads and a clamping surface facing the lead slot, a clamping bar disposed within the lead slot opposite the clamping surface, and a threaded screw coupled to the clamping bar, wherein the retainer is configured so that rotation of the screw moves the clamping bar laterally toward or away from the clamping surface of the disc; and
a cover configured to be disposed over the retainer and removably coupled to the plug base for lead retention.

15. The burr hole plug of claim 14, wherein the retainer further comprises a disc tab extending from the disc and a clamp tab extending from the clamping bar, wherein the threaded screw extends from the disc tab to the clamp tab.

16. The burr hole plug of claim 15, wherein the disc tab defines a non-threaded aperture and the clamp tab defines a threaded aperture, wherein the threaded screw extends through the non-threaded aperture of the disc tab and the threaded aperture of the clamp tab.

17. The burr hole plug of claim 14, wherein the clamping bar comprises a threaded aperture and the threaded screw extends into the threaded aperture of the clamping bar.

18. The burr hole plug of claim 17, wherein the retainer further comprises a screw tab extending from the disc, wherein the screw tab comprises a non-threaded aperture and the screw extends through the non-threaded aperture of the screw tab.

19. A burr hole plug kit, comprising:
the burr hole plug of claim 14; and
a tool configured for rotating the screw.

20. A method for clamping at least one lead within the burr hole plug of claim 14, the method comprising:
positioning the plug base in the burr hole of the skull of the patient with the at least one lead extending out of the burr hole;
disposing the retainer in the plug base with the at least one lead extending through the lead slot defined by the disc of the retainer; and
rotating the screw to move the clamping bar laterally toward the clamping surface of the disc of the retainer to clamp the at least one lead between the clamping bar and the clamping surface.
